(19) United States
(12) Patent Application Publication
(10) Pub. No.: US 2004/0123337 A1
(43) Pub. Date: Jun. 24, 2004

(54) Method for treating an embryo

(76) Inventor: Norbert Gleicher, Chicago, IL (US)

Correspondence Address: BEEM PATENT LAW FIRM, 53 W. JACKSON BLVD., SUITE 1352, CHICAGO, IL, 60604-3787, US

(21) Appl. No.: 10/326,945

(22) Filed: Dec. 19, 2002

(51) Int. Cl. A01K 67/027

(52) U.S. Cl. 800/21

(57) ABSTRACT

An inventive method is disclosed for treating an embryo by creating a chimera, wherein blastomeres from a donor embryo at an early stage is removed and introduced into a recipient embryo at an early stage. The recipient embryo is then grown to a later stage of development. Approximately one to three blastomeres are removed from the donor embryo at Day 3 of development and introduced into a recipient embryo at Day 3. Incorporation of the donor blastomeres into the recipient embryo is determined at a later stage of embryonic development.

### BACKGROUND

1. Field of the Invention

The present invention relates to a method for treating an embryo by creating a chimera.

2. Background of the Invention

In vitro fertilization (IVF) is a process that creates embryos by joining a sperm with an oocyte under laboratory conditions. When fertilization is difficult or impossible naturally, IVF provides a way to produce viable embryos capable of transfer into the uterus for development.

In vitro embryos can be screened for genetic defects by preimplantation genetic diagnosis (PGD). Some embryos created through IVF cannot be transferred into the uterus. Embryos can be unsuitable for transfer into the uterus because they carry a genetic disease. Single gene disease is one type of affliction resulting in unhealthy embryos.

A. Single Gene Defects

Approximately 3,000 human genetic diseases are believed to be the consequence of single gene defects (Sachs and Uorf, 81 Obstet. Gynecol., 485 (1993)). Various methods have been utilized in attempts to introduce genes into affected cells to correct the genetic defect (Stribley, et al., 77 Fertil. Steril., 645 (2002)). Somatic gene therapy involves the introduction and expression of recombinant genes into somatic cells for the purpose of ameliorating a disease. Corrective gene therapy protocols aim to transfer genes directly to the diseased cell type to correct the genetic defect.

Implementation of gene therapy may be achieved through either an ex vivo or in vivo approach. The ex vivo approach involves the harvesting and cultivation of cells from patients after surgical biopsy or organ resection. The cells are then cultured in vitro and treated for gene transfer. Finally, the transfected cells are reintroduced back into the patient. The effectiveness of the ex vivo approach depends upon the permanent introduction of the recombinant genes into the target cell, which elicits permanent expression of the therapeutic gene product.

The in vivo approach to gene therapy involves the direct administration of the transgene into the target organ or tissues. An embryo that is suited to the in vivo approach must be in a late stage of development after organs and tissues have formed. In vivo techniques include the direct injection of DNA into tissues such as muscle, thyroid, and joint; coupling of DNA to proteins with specific receptors on the target cell; or the merging of DNA with solid particles that are injected into the cell.

Such experimental gene therapy treatments suggest that, in order to ameliorate the disease, the repair of a genetic defect does not have to be complete (Hacien-Bey-Abina, et al., 346 N. Engl. J. Med., 1185, (2002)). Although the genetic repair in this case of combined immunodeficiency is incomplete, it can be sufficient to provide protective immunity.

B. Chimeric Embryos

The creation of chimeric embryos is a manipulation of embryos resulting in the incorporation of more than one genetic identity into one embryo. When the chimeric embryo develops to later stages, the embryo will express the gene products from the distinct genetic identities incorporated.

Chimeric embryos can be created by introducing embryonic stem (ES) cells derived from the inner cell mass (ICM) of blastocysts into the blastocoel cavity of different blastocysts (Bradley, et al., 309 Nature, 255-256, (1993), Wood, et al., 90 Develop Bio, 4582-4585, (1993)). ES cells possess the ability to proliferate indefinitely in an undifferentiated state, and are capable of contributing to the formation of normal tissues and organs of a chimeric individual when injected into a recipient embryo. Since ES cells are pluripotent, the introduction of these cells with exogenous genetic information does not impair the ability of these cells to incorporate into the germ line (Gossler, et al., 83 Proc. Natl. Acad. Sci. 9065-9069, (1986)). Injection of ES cells derived from the ICM into blastocysts is effective at creating chimeric embryos.

Another method of creating chimeric embryos is by aggregating blastomeres from several embryos (Alikani and Willadsen, 5 Reprod. Biomed., 56-58, (2002)). Healthy blastomeres from non-viable human embryos were aggregated and cultured into a later stage. This method of aggregating healthy blastomeres from various non-viable embryos allows some viable blastocysts to develop from otherwise non-viable embryos.

When utilizing in vitro fertilization for research and potential implantation purposes, viable and genetically approved normal embryos are needed. Afflicted embryos may not meet standards for transfer into the uterus.

### SUMMARY OF THE INVENTION

The present invention is directed to methods for treatment of an early embryo by creating a chimera. The chimera is created by introducing blastomeres into an early stage embryo. Blastomeres are removed from a donor embryo and introduced into a recipient embryo. The recipient embryo is then grown to a later stage. The recipient embryo becomes a chimera upon incorporation of the blastomeres.

In one method, the blastomeres removed from the donor embryo number between one and three. The blastomeres are taken out of the donor embryo at an early stage of the embryonic development, ideally three days after fertilization. Blastomeres, numbering between one and three, are then transferred to a recipient embryo that is also in early embryonic development, ideally an embryo that is three to four days after fertilization. The blastomeres introduced into the recipient embryo can comprise from about 12% to about 40% of the total blastomeres in the recipient embryo after the introducing step. Ideally, the blastomeres introduced into the recipient embryo will comprise from about 25% to about 40% of the total blastomeres in the recipient embryo.

In another aspect of the invention, in order to determine incorporation of the donor embryo into the developing recipient embryo, the donor embryo and the recipient embryo are different genders. The donor embryo can be male. The recipient embryo can be female. After the introduction of the blastomeres into the recipient embryo and the recipient embryo has grown to a later stage, the degree of blastomere incorporation can be determined by identifying chromosome x and chromosome y in the cells of the recipient embryo. One possible later stage to which the recipient embryo is grown is the blastocyst stage, typically between five and six days after fertilization.

In another aspect of the invention, the removing step and the introducing step are accomplished with pipettes. The pipettes utilized in both steps are the holding pipette for holding the embryo and the biopsy pipette for manipulating the blastomeres.

In still another aspect of the invention, the donor embryos and the recipient embryos used can be animal, mammal, as well as human embryos. This method for embryo treatment can be used to treat genetic defects in the recipient embryo.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 shows a schematic diagram of the invention.

FIG. 2 shows a schematic diagram for a possible use of the invention in treating genetically defective embryos.

FIG. 3 shows xy-donor embryo on Day 6 after donating 3 blastomeres on Day 3.

FIG. 4 shows xx-recipient embryo on Day 5 after 1 cell transplant at 7-cell stage on Day 3.

FIG. 5 shows xx-recipient embryo on Day 6 after 3 cell transplant in 6-cell embryo on Day 3.

FIG. 6 shows xx-recipient embryo on Day 5 after receiving 2 cell transplant in 4-cell embryo on Day 3.

FIG. 7 shows xx-recipient embryo on Day 5 after 1 cell transplant in 5-cell embryo on Day 3.

FIG. 8 shows xx-recipient embryo on Day 6 after receiving 2 cell transplant in 6-cell embryo on Day 3.

FIG. 9 shows xx-recipient embryo on Day 6 after receiving 3 cell transplant in 5-cell embryo on Day 3.

FIG. 10 shows xx-recipient embryo on Day 6 after receiving 1 cell transplant in 7-cell embryo on Day 3.

FIG. 11 shows xx-recipient embryo on Day 6 after receiving 2 cell transplant in 4-cell embryo on Day 3.

FIG. 12 shows xx-recipient embryo on Day 5 after receiving 2 cell transplant in 3-cell embryo on Day 3.

### DETAILED DESCRIPTION OF THE INVENTION

To investigate the acceptance of transplanted blastomeres into recipient embryos, a chimeric model is utilized: blastomeres are taken from donor embryos and introduced to recipient embryos and the recipient embryos are grown and analyzed for chimeric qualities. The donor embryos and the recipient embryos must be at an early stage in the embryonic development, i.e. before cell differentiation.

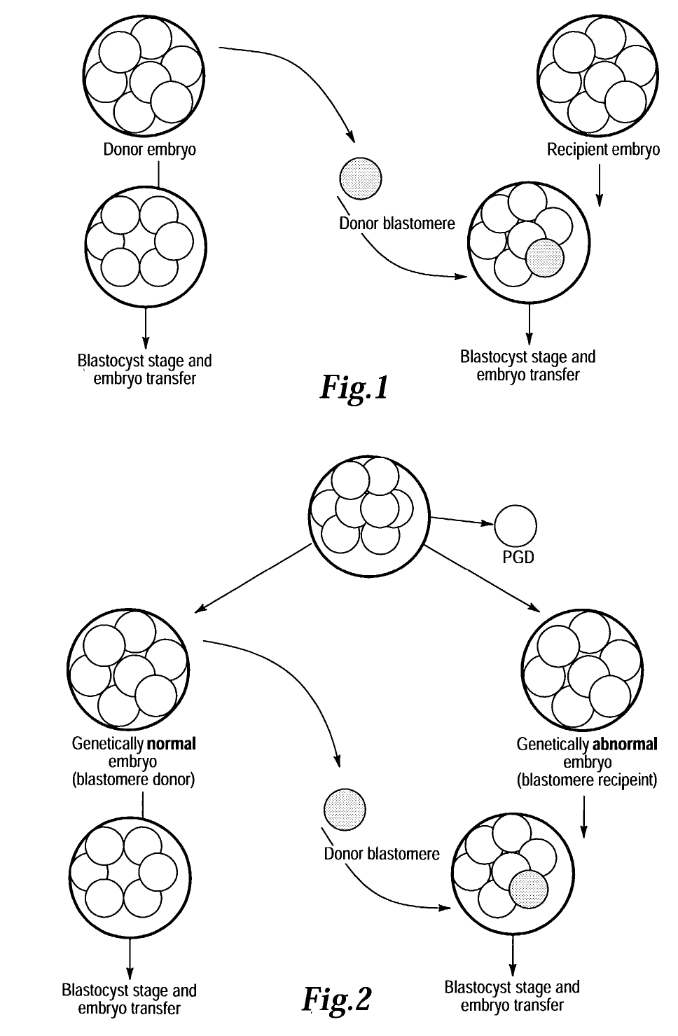

FIG. 1 shows the steps of extracting blastomeres from a donor embryo and introducing the blastomeres to a recipient embryo. At least one blastomere is taken out of a donor embryo and introduced to a recipient embryo. Both the donor embryo and the recipient embryo are viable after the blastomere transplantation step.

Donor embryos and recipient embryos previously cryopreserved on day 3 (4-10 cell stage) are thawed. The embryos are air thawed for 30 seconds. Then they undergo a 40-50 second thaw in a 30° C. water bath. The embryos are then placed in 1.0M PROH/0.2M sucrose for five minutes and then in 0.5M PROHO/0.2M sucrose (PROHO from GibbcoBRL, Grand Island, N.Y.; sucrose from Sigma, St. Louis, Mo.) for five minutes. Embryos are then transferred to 0.2M sucrose Gibco ET (Embryo Transfer) Freezing Medium for 10 minutes. Place embryos in room temperature Gibco ET Freezing Media, then place on 37° C. warmer for 10 minutes to allow gradual warming.

Alternatively, freshly produced embryos can also be used. The thawed embryos are then placed in a drop of biopsy medium under mineral oil by holding them with a holding pipette. The zona pellucida is locally digested, by releasing acidified Tyrode's solution (Sigma, St. Louis, Mo. 63178) through one assistant hatching pipette. During the blastomere removal process the blastomeres are removed from donor embryos through a biopsy opening in zona pellucida, and the donor embryo put back into culture (culture consists of IVC—Three Blastocyst Medium with 5% Human Serum Albumin (In VitroCare Inc., San Diego, Calif.)). One to three blastomeres are removed from each donor embryo.

Blastomeres are then picked up with the biopsy pipette and gently inserted through the initial biopsy opening in the zona pellucida of recipient embryo in a blastomere introduction process. One to three blastomeres are introduced into recipient embryo during the blastomere introduction process. Once blastomere introduction process has taken place, the recipient embryo is returned to culture. Recipient embryos are uniformly cultured to day 5 or 6. On day 5 or 6, normal embryos reach blastocyst stage. Later on day 5, or on day 6, normal embryos start hatching. Later on day 6, some embryos can be observed until fully hatched.

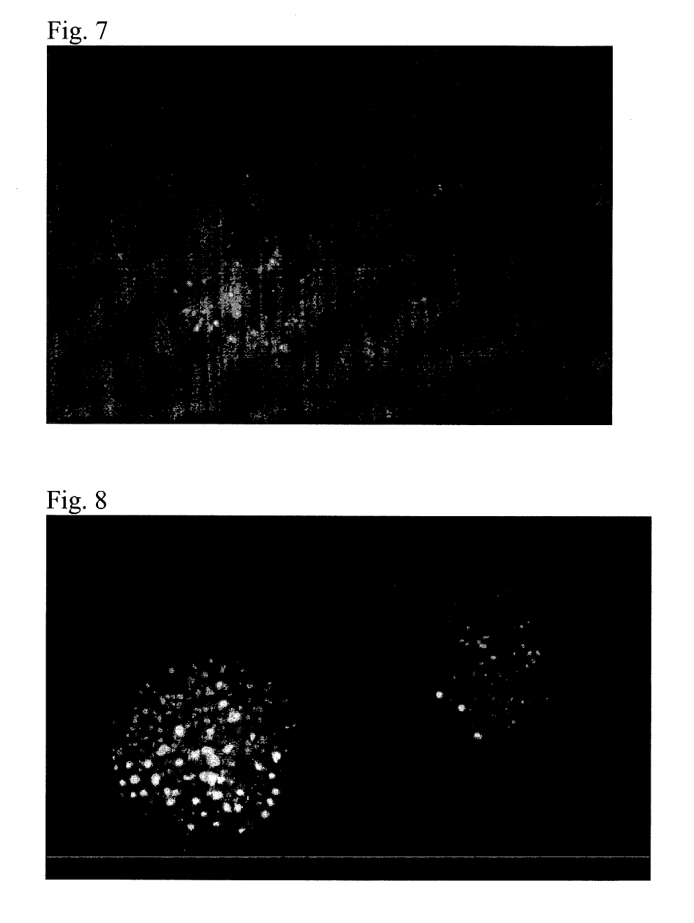

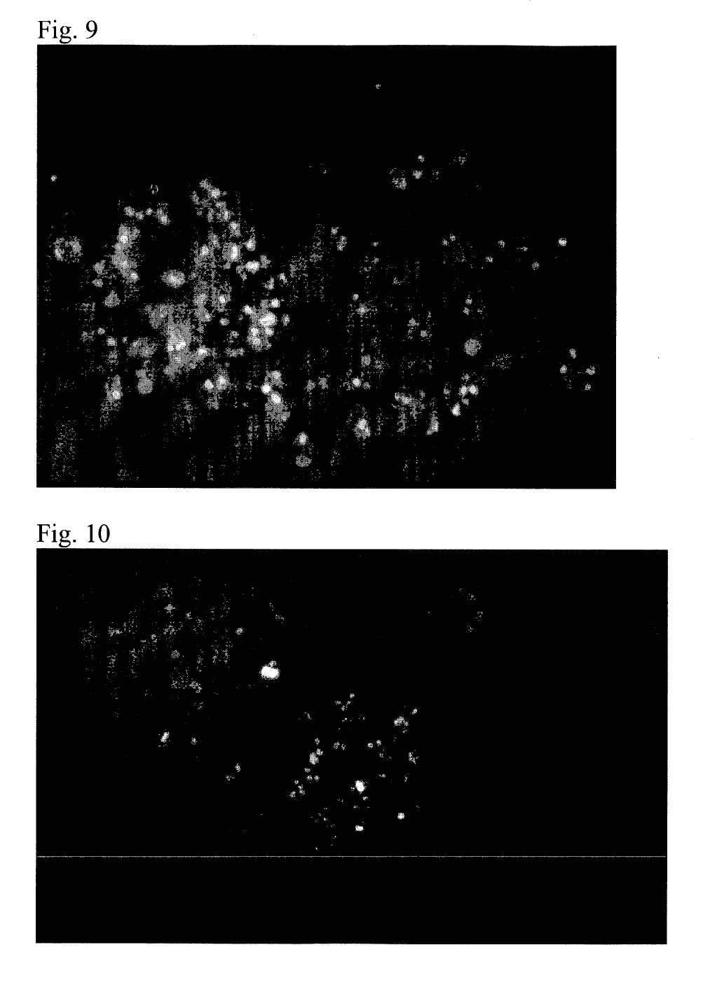

As can be seen in EXAMPLE 1 below, the fact that the transplanted blastomeres demonstrate an apparently normal distribution of daughter cells throughout the inner cell mass and trophoectoderm suggests that these transplanted blastomeres are integrated into the early development of recipient embryos. The apparently highly successful grafting of blastomeres into developing recipient embryos supports the contention that early embryos are amenable to this type of treatment. The amount of y-signal increases with an increasing number of blastomeres transferred, suggesting that the amount of potential gene therapy can be titrated. As FIG. 4, FIG. 7, and FIG. 10 illustrate, among embryos that developed to blastocyst stage, there is minor donor blastomere incorporation when the donor blastomeres consisted of 16.7% or less of the blastomeres in the recipient embryo after transplantation. FIG. 5, FIG. 8, and FIG. 9 show a profound incorporation of the donor blastomeres at the blastocyst stage when the donor blastomeres consist of 25% or more of the blastomeres in the recipient embryo after transplantation.

These results offer the possibility of blastomere transplantation as a treatment tool. Preimplantation genetic diagnosis (PGD) is increasingly utilized to diagnose embryos affected by genetic diseases. The potential outcome will always result in embryos being considered “affected,” “normal,” or “undetermined” (i.e. no clear diagnosis can be reached due to unavoidable technical difficulties with PGD). Under current practice guidelines, only normal embryos are transferred to the uterus during in vitro fertilization, with embryos in the other two groups being discarded. Often, this leaves few or no embryos for transfer.

In FIG. 2, the blastomere transplantation is depicted as a potential method for treating a genetically diseased embryo. The embryo may be any type of animal embryo, including mammalian and human. A blastomere is removed from each one of day 3 embryos to undergo PGD. Blastomeres, numbering between one and three, will be removed from a genetically normal embryo and introduced into a genetically abnormal embryo. The treatment can utilize gender-matched embryos as donor embryo and recipient embryo. The donor embryo and the recipient embryo can be sibling embryos or unrelated embryos. The donor embryo and the recipient embryo are both grown to later stages. A few healthy donor blastomeres that incorporate into the recipient embryo and produce a normal gene product can titrate the effect of the genetic disease in the recipient embryo. The titration of the genetic disease can potentially result in an embryo appropriate for transfer into the uterus.

The treatment method can be used to develop animals possessing more than one desirable trait. For example, the embryo of a cow that is genetically predisposed to produce a large amount of milk can receive blastomeres from a cow that is genetically predisposed to growing large amounts of meat. Or vice versa, an embryo genetically predisposed to growing large amounts of meat could receive blastomeres genetically predisposed to producing large amounts of milk. The resulting chimera could potentially grow into a cow that produces large amounts of milk as well as large amounts of meat. The resulting animal would be suitable for both dairy and meat production.

Blastomere transplantation can turn single gene afflicted embryos into viable embryos capable of transfer into the uterus. The ability to treat afflicted embryos allows more fertilized embryos to be transplanted into the uterus. Genetically afflicted embryos, which do not meet the standards for transfer, can now be treated and considered for transfer into the uterus. The ability to treat affected embryos can also greatly increase the efficiency of IVF by not requiring the fertilization of large numbers of oocytes, consequently, making IVF more cost-effective. A wider utilization of PGD combined with blastomere transplantation in an effort to prevent single gene diseases can have a significant public health impact. Embryos diagnosed with a single gene defect are not necessarily precluded from transfer to the uterus.

In summary, it is shown that a method for treating an early embryo comprises the steps of removing at least one blastomere from a donor embryo at an early stage, introducing at least one of said blastomeres into a recipient embryo at an early stage, and growing the recipient embryo to a later stage. The blastomeres removed from the donor embryo number between one and three. The donor embryo from which the blastomeres are removed is in an early stage at three days after fertilization. Likewise, the recipient embryo into which the blastomeres are introduced is at an early stage at three to four days after fertilization. The blastomeres introduced into the recipient embryo number between one and three. The blastomeres are removed from the donor embryo by pipettes, a holding pipette and a biopsy pipette. The blastomeres are introduced into the recipient embryo by pipettes, a holding pipette and a biopsy pipette.

The donor and recipient embryos involved in the treatment method can be animal, mammal, or human embryos. This method can be used to treat genetic diseases in recipient embryos.

### EXAMPLE 1

### Creating Chimeric Embryos

To investigate the acceptance of transplanted blastomeres into recipient embryos, a chimera model is utilized: human male embryos, with an xy-chromosomal complement, serve as blastomere donors, while human female embryos, with an xx-chromosomal complement, serve as recipients. Integration of xy-blastomeres into the recipient xx-embryo is then traced by staining the day 5 or 6 blastocyst stage embryo with a y-chromosome marker, as is routinely done in PGD studies for the determination of embryonic gender.

### Materials and Methods

Forty four embryos previously cryopreserved on day 3 (4-10 cell stage) were thawed in routine fashion. Thirty seven embryos (84%) survived the thaw and underwent embryo biopsy for PGD, as previously described, in order to determine the gender of each embryo. Specifically, the biopsy is performed on a Nikon Diaphot (Nikon, Inc., Melville, N.Y. 11747) inverted microscope, using Navishige manipulators (Navishige International USA, Inc., East Meadow, N.Y. 11554), with microtools from Humagen (Humagen Fertility Diagnostics, Inc., Charlottesville, Va. 22911).

Day 3 embryos at 4- to 10-cell stage are placed in a drop of biopsy medium under mineral oil by holding them with a holding pipette. The zona pellucida is locally digested, by releasing acidified Tyrode's solution (Sigma, St. Louis, Mo. 63178) through the assistant hatching pipette. Through a small opening created, one blastomere is aspirated and removed from the embryo by micromanipulation, using a biopsy pipette. The embryo is put back to culture and the blastomere's gender is determined by fluorescent in situ hybridization (FISH). (See Handyside, Rontogianni, et al. Nature Med. 344, 768 (1990)).

To perform FISH, the blastomere is placed in a drop of hypotonic solution for 5 minutes and transferred to a glass slide. Carnoy's fixative solution (Sigma, St. Louis, Mo. 63178) (3:1 methanol and acetic acid) is then dropped onto the blastomere cell, while under an inverted microscope. All slides are dehydrated through an ethanol series (70%, 85%, 100% ethanol) and then air dried.

Probes CEP Y (DYZI, satellite III spectrumGreen) and CEP X (DXZI, alpha satellite spectrumOrange) are used to identify chromosome y and x, respectively (Vysis, Inc., Downers Grove, Ill. 60515). The two colour hybridization solutions are applied to the target area and a coverslip is placed. Embryonic DNA is denatured with the probe mixture at 78° C. for 10 minutes, followed by hybridization at 37° C. for 2 hours in a HYBrite unit (Vysis, Inc., Downers Grove, Ill. 60515).

Slides are washed with 0.4% Saline Sodium Citrate (SSC)/0.3% Nonidet P-40 (NP40) at 73° C. for 5 minutes in a water bath and 2×SSC/0.1% NP40 (2×SSC solution containing 0.1% NP-40) for 1 minute at room temperature, air dried and a counter stain (DAPI II). (Vysis, Inc., Downers Grove, Ill. 60515) is applied. Specific signals are viewed and documented under the Olympus Ax70 fluorescence microscope with appropriate filter sets. Images are captured using a DKC-5000 digital camera (Scientific, Inc., Middlebush, N.J. 08875).

After the gender of an embryo is determined, male embryos, serving as blastomere donors, are placed into a drop of biopsy medium, covered by mineral oil and held in place with a holding pipette. The embryo is then touched gently with a biopsy pipette to find the earlier established biopsy site. Additional blastomeres are then removed through the original biopsy opening in the zona pellucida, in the same fashion as previously described and the embryo is put back into culture.

Female embryos are treated similarly, though they serve as recipients. Female embryos, too, are placed into a drop of biopsy medium, covered by mineral oil and the prior biopsy site was identified. Blastomeres are then picked up with the biopsy pipette and gently inserted through the initial biopsy opening in the zona pellucida of the recipient embryo in a step reversing the prior blastomere removal process. Once the blastomere transplant has taken place, the embryo is returned to routine culture.

Recipient embryos are uniformly cultured to day 5 or 6, while donor embryos are only selectively cultured to days 5 or 6, to confirm persistent viability after removal of up to 3 blastomeres (FIG. 2). On day 5, normal embryos reach blastocyst stage. Later on day 5, or early on day 6, normal embryos start hatching. Later on day 6, some embryos are observed until fully hatched. Embryos are then, at all of those blastocyst stages, investigated by FISH, for the presence of x- and y-chromosomes, utilizing the same techniques as previously described for the gender determination of individual blastomeres.

In the here utilized chimera model, following blastomere transplantation, the appearance of a green marker (y-chromosome) against a red background (x-chromosome) represents one half of the donor genotype accepted and integrated by the recipient embryo, since the other half (the x-chromosome) cannot be differentiated against the recipient embryo's own chromosomal background. Y-penetration is classified as negative (no signal), minor (1-15 signals), moderate (16-30 signals) and profound (over 31 signals) for each blastocyst stage embryo.

Recipient embryos receive either 1, 2 or 3 blastomeres. Donor embryos are either completely dismantled, with all blastomeres used for transplantation, or have 1 to 3 blastomeres removed (beyond the original blastomere removed for gender determination) when they are cultured to blastocyst stage (FIG. 2). Sixteen male embryos served as donor embryos and 21 female embryos received blastomere transplants.

### Results

Amongst recipient embryos, 4 (19%) arrested their development before reaching blastocyst stage. However, only 12 (57%) are considered to have reached normal blastocyst stage. Nine embryos were allowed to hatch either partially or completely, before staining.

TABLE 1 summarizes the intensity of y-signal in transplant recipient embryos. As can be seen, the number of blastomeres transferred generally appears to correlate with the intensity of y-penetration into the embryo.

TABLE 1
Correlation between y-signal intensity
and number of blastomeres transferred

| Number of | | | | |
|---|---|---|---|---|
| blastomeres | Number of | Blastocyst | Number | Penetration |
| transplanted | embryos | quality | y-signals | grading |
| 1 | 3 | Poor | 3 | Minor |
| | | Poor | 22 | Moderate |
| | | Good | 14 | Minor |
| 2 | 16 | Excellent | >50 | Profound |
| | | Excellent | >50 | Profound |
| | | Good | 11 | Minor |
| | | Poor | 18 | Moderate |
| | | Arrested | 12 | Minor |
| | | Excellent | >60 | Profound |
| | | Excellent | >50 | Profound |
| | | Good | 14 | Minor |
| | | Good | 12 | Minor |
| | | Good | 11 | Minor |
| | | Good | 12 | Minor |
| | | Poor | 0 | Negative |
| | | Poor | 0 | Negative |
| | | Arrested | 3 | Poor |
| | | Arrested | 9 | Poor |
| | | Arrested | 7 | Poor |
| 3 | 2 | Excellent | >90 | Profound |
| | | Good | 38 | Profound |

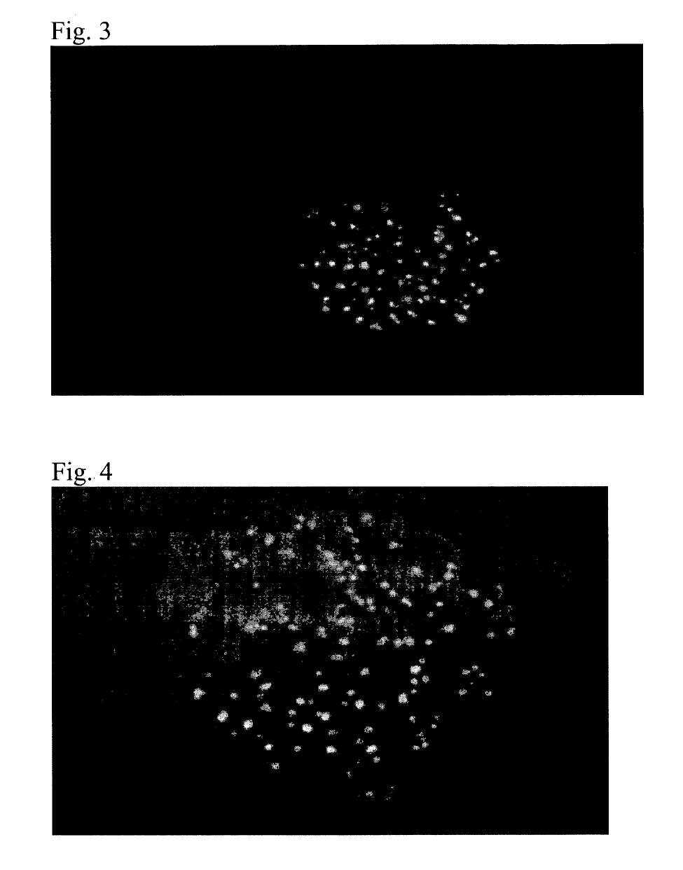

In FIG. 3, the donor embryo is clearly viable after donating two blastomeres. The xy-donor embryo had 1 blastomere removed for gender determination early on day 3, and then had two more blastomeres removed for transplantation to a recipient embryo. It developed normally to a day 6 blastocyst stage embryo and is shown hatching. Red signal reflects x-chromosome; green signal reflects y-chromosome.

FIG. 4 demonstrates a normally developing day 5 blastocyst stage recipient embryo. This xx-embryo received on day 3, at 7-cell stage, a 1-cell xy-blastomere transplant. The recipient embryo now demonstrates a symmetric dissemination of the y-signal throughout the inner cell mass and trophectoderm. The anatomy of this blastocyst stage embryo is recognizable, because of counterstaining of nuclei with DAPI II (blue color). The y-signal in this embryo is classified as minor, with only 14 signals. DAPI II staining step involves applying 3 microliters of DAPI II counterstain (125 nanograms of DAPI/ml in antifade mounting solution (Vysis, Downers Grove, Ill.)). onto the target area and covering with a coverslip, the nucleus will be identified under the DAPI single bandpass filter (Vysis).

FIG. 5 demonstrates a completely hatched recipient embryo on day 6. Once again, the evidence of a y-chromosome can be seen throughout the recipient embryo. This embryo received 3 donor blastomeres as a 6-cell embryo on day 3. Approximately 33% of the blastomeres in the recipient embryo after transplantation were donor blastomeres. The penetration by y-marker was classified as profound, since over 90 y-signals were counted.

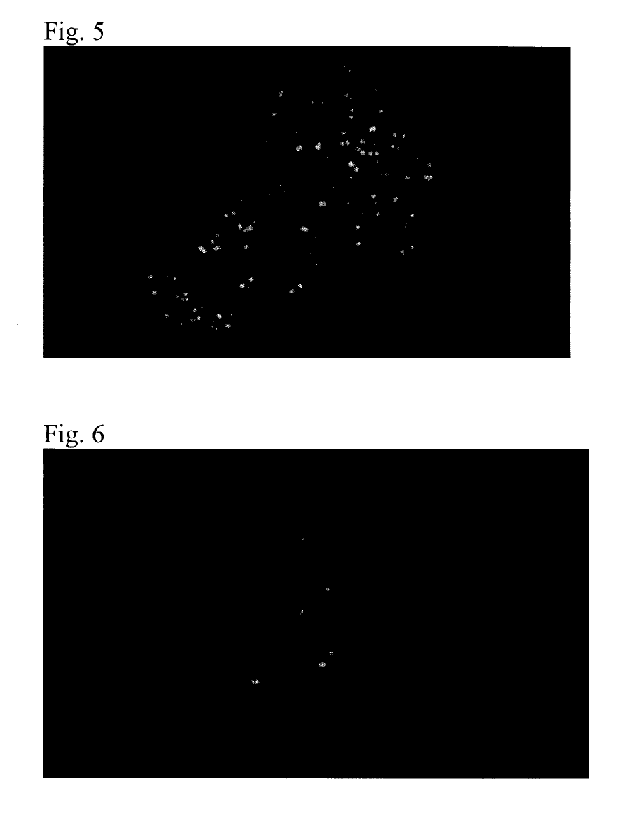

FIG. 6, in contrast, demonstrates uneven sporadic y-staining in an abnormally developing embryo, which arrested on day 5 at morula stage. This embryo demonstrates only a minor degree of y-signal. Only 3 signals were counted after transplantation of 2 donor blastomeres on day 3 into a 4-cell embryo, which had been judged to be of relatively poor quality, since it demonstrated a considerable degree of blastomere fragmentation. Even though 50% of the blastomeres in the recipient embryo are donor blastomeres, the poor quality of the embryo hindered the blastomere incorporation.

FIG. 7 shows a day 5 blastocyst stage embryo after transplantation of 1 blastomere on day 3 into a 5-cell embryo. The percentage of donor blastomeres in the recipient embryo after transplantation is 16.7%. There is a minor degree of y-signal, since only 11 signals were detected.

FIG. 8 shows a day 6 blastocyst stage embryo, which has completely hatched and fractured into two pieces. This embryo received 2 donor blastomeres on day 3, as a 6-cell embryo. The percentage of donor blastomeres in the recipient embryo after transplantation is 25%. Y-penetration is profound, with over 50 signals counted.

FIG. 9 shows a day 6 blastocyst after hatching, with nuclei counterstained with DAPI II. This embryo received on day 3, at 5-cell stage, a transplant of 3 donor blastomeres. The percentage of donor blastomeres in the recipient embryo after transplantation is 37.5%. The penetration of the y-signal is profound, with 38 signals counted.

FIG. 10 shows a day 6 blastocyst stage embryo after hatching and of poor quality. Nuclei are counterstained with DAPI II. This embryo received a 1-blastomere transplant on day 3, as a 7-cell embryo. The percentage of donor blastomeres in the recipient embryo after transplantation is 12.5%. The y-signal distribution is minor, with only 3 signals detected.

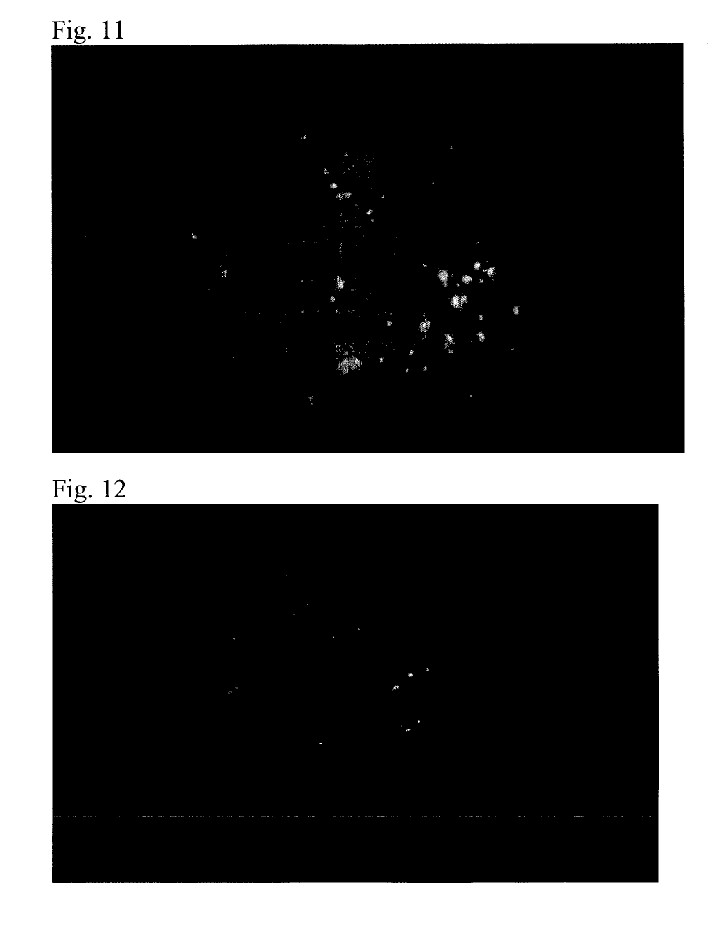

FIG. 11 shows a day 6 embryo arrested at morula stage. This embryo received a 2-blastomere transplant on day 3 as a 4-cell embryo. The percentage of donor blastomeres in the recipient embryo after transplantation is 33%. The y-signal distribution is minor, with 12 signals detected. The embryo did not proceed until blastocyst stage like most healthy embryos and thus the data is difficult to analyze.

FIG. 12 shows a day 5 embryo arrested at morula stage. This embryo received a 2-blastomere transplant on day 3, when at 3-cell stage. The percentage of donor blastomeres in the recipient embryo after transplantation is 40%. The y-signal distribution is minor, with 7 signals detected. The embryo arrested prior to blastocyst stage, making the data difficult to compare with other blastocyst-stage embryos.

Twelve normally developing embryos uniformly demonstrate an even and well-distributed staining pattern for the y-chromosome, while nine abnormally developing or arrested embryos demonstrate an uneven, spotty and usually diminished staining pattern. Indeed, among such embryos, only 3 (33%) demonstrate a y-distribution pattern that was considered normal.

As shown in EXAMPLE 1, the blastomeres introduced into the recipient embryo comprise from about 12% to about 40% of the total blastomeres in the recipient embryo after the introduction of the blastomeres. Preferably, the blastomeres introduced into the recipient embryo comprise from about 25% to about 40% of the total blastomeres in the recipient embryo after the introduction step.

The donor embryo is male and the recipient embryo is female. The incorporation of the donor blastomeres into the recipient embryo can be determined in a later stage. The determination step can identify the amount of x and y chromosomes in the recipient embryo at a later stage, between day 5 and day 6 after fertilization.

The present invention is not limited to the above-described embodiments and methods, but should be limited solely by the following claims.

We claim:

1. A method for treating an early embryo by creating a chimera, comprising the steps of:
removing at least one blastomere from a donor embryo at an early stage;
introducing at least one of said blastomeres into a recipient embryo at an early stage;
growing said recipient embryo to a later stage.

2. A method according to claim 1, wherein said blastomeres removed from said donor embryo number between one and three.

3. A method according to claim 1, wherein said early stage of said donor embryo is three to four days after fertilization.

4. A method according to claim 1, wherein said early stage of said recipient embryo is three days after fertilization.

5. A method according to claim 1, wherein said blastomeres introduced into said recipient embryo number between one and three.

6. A method according to claim 1, wherein said blastomeres introduced into said recipient embryo comprise from about 12% to about 40% of total blastomeres in recipient embryo after said introducing step.

7. A method according to claim 6 wherein said blastomeres introduced into said recipient embryo comprise from about 25% to about 40% of total blastomeres in recipient embryo after said introducing step.

8. A method according to claim 1, wherein said donor embryo is male.

9. A method according to claim 1, wherein said recipient embryo is female.

10. A method according to claim 1, further comprising determination of incorporation of said introduced blastomeres into said recipient embryo.

11. A method according to claim 10, wherein said determining step comprises identifying chromosome x and chromosome y.

12. A method according to claim 1, wherein said later stage is a blastocyst stage between 5 and day 6 after fertilization.

13. A method according to claim 1, wherein said removing step is accomplished with pipettes.

14. A method according to claim 13, wherein said pipettes include a holding pipette and a biopsy pipette.

15. A method according to claim 1, wherein said introducing step is accomplished with pipettes.

16. A method according to claim 15, wherein said pipettes include a holding pipette and a biopsy pipette.

17. A method according to claim 1, wherein said donor and recipient embryos are animal embryos.

18. A method according to claim 1, wherein said donor and recipient embryos are mammalian embryos.

19. A method according to claim 1, wherein said donor and recipient embryos are human embryos.

20. A method according to claim 1, wherein said method is used to treat genetic diseases in said recipient embryo.
